(19) United States
(12) Patent Application Publication
(10) Pub. No.: US 2017/0349693 A1
(43) Pub. Date: Dec. 7, 2017

(54) INNOVATIVE HYDROPHILIC POLYISOCYANATES WITH IMPROVED STORAGE STABILITY

(71) Applicant: EVONIK DEGUSSA GMBH, Essen (DE)

(72) Inventors: Emmanouil Spyrou, Schermbeck (DE); Evelyn Albrecht, Recklinghausen (DE); Iris Brueckner, Dorsten (DE); Annegret Lilienthal, Dorsten (DE); Wiebke Stache, Herten (DE); Judith Schoder, Haltern am See (DE)

(73) Assignee: EVONIK DEGUSSA GMBH, Essen (DE)

(21) Appl. No.: 15/681,954

(22) Filed: Aug. 21, 2017

Related U.S. Application Data
(63) Continuation of application No. 14/431,085, filed on Mar. 25, 2015.

(30) Foreign Application Priority Data
Oct. 4, 2012 (DE) 102012218081.8

(51) Int. Cl. C08G 18/78; C09D 175/08; C08L 75/08; C09J 175/08; C07D 229/00

(52) U.S. Cl. CPC C08G 18/7887; C09J 175/08; C07D 229/00; C08L 75/08; C09D 175/08

(57) ABSTRACT

The invention relates to innovative, water-dispersible, hydrophilic polyisocyanates and polyisocyanate mixtures, to a process for preparing them, to compositions comprising the hydrophilic polyisocyanates of the invention, and to their use as a starting component in the production of polyurethane plastics, more particularly as crosslinkers for water-soluble or water-dispersible film-forming, adhesives or sealant binders or binder components having groups which are reactive towards isocyanate groups.

The invention relates to innovative, water-dispersible, hydrophilic polyisocyanates and polyisocyanate mixtures, to a process for preparing them, to compositions comprising the hydrophilic polyisocyanates of the invention, and to their use as a starting component in the production of polyurethane plastics, more particularly as crosslinkers for water-soluble or water-dispersible film-forming, adhesives or sealant binders or binder components having groups which are reactive towards isocyanate groups.

Against the background of an increasingly more stringent environmental legislation, recent years have seen an increase in importance of water-dispersible polyisocyanates for various application fields. They presently find use in particular as crosslinker components for water-thinnable two-component polyurethane paints (2K PU paints) at a high quality level, or as additives for aqueous dispersion-based adhesives. They serve for the crosslinking of aqueous dispersions in the finishing of textiles and of leather, or of formaldehyde-free textile printing inks, and are also suitable, furthermore, for example, as wet strengthening auxiliaries for paper, as disclosed in EP 0 959 087, for example.

For the preparation of water-dispersible hydrophilic polyisocyanates there are a host of different processes known, with a part being played by ionic modifications, among others. EP-A 0 443 138, EP-A 0 510 438 and EP-A 0 548 669, for example, describe polyisocyanate mixtures which contain chemically bonded carboxyl groups.

Although such polyisocyanates can be incorporated very finely by stirring into aqueous systems, following neutralization of the carboxyl groups, without the need for high shearing forces, their storage stability, particularly in neutralized form, is nevertheless wholly inadequate.

EP 0 703 255 62 describes ionically hydrophilized, water-emulsifiable polyisocyanates which as emulsifiers have reaction products from the reaction with 2-hydroxyethanesulphonic acid or 3-hydroxypropanesulphonic acid. These hydrophilizing agents, however, have a range of disadvantages. Hydroxypropanesulphonic acid, for example, is in equilibrium with its anhydride, 1,3-propane sultone, which is classed as a carcinogen. Especially on the industrial scale, therefore, it can be utilized exclusively in the form of aqueous solutions and is therefore fundamentally unsuitable as a synthesis component for the preparation of modified polyisocyanates. Furthermore, EP 0 703 255 B2 makes no comment on the stability or the processing window (“pot life”) of emulsions prepared from the hydrophilic polyisocyanates.

EP 1 287 052 61 discloses modified polyisocyanates obtainable by reacting polyisocyanates with 2-(cyclohexylamino)ethanesulphonic acid and/or 3-(cyclohexylamino)-propanesulphonic acid in the presence of a suitable neutralizing agent, giving pale-coloured, storage-stable products. In relation to the storage stability, however, all that is disclosed is that the aqueous emulsions obtainable from the hydrophilic modified polyisocyanates were still fully stable after a standing time of 5 hours. They showed neither visible CO₂ evolution nor instances of precipitation or sediment. No statement is made, however, concerning the actual NCO content or the reactivity of the system after 5 hours.

Used presently in practice for the great majority of applications are exclusively nonionic polyisocyanates which are hydrophilically modified using polyethers. The preparation of water-dispersible polyisocyanates of this kind is discussed comprehensively in EP 0 959 087, EP 0 206 059 and EP 0 540 985, for example. Despite their widespread use for a whole host of different applications, however, polyisocyanates modified with nonionic polyethers have a series of fundamental disadvantages. During dispersing, they can in many cases be incorporated homogeneously into aqueous media only with employment of considerable shearing forces. Moreover, the high polyether content, which is necessary for sufficient dispersibility particularly in the context of their use as crosslinkers in aqueous 2K-PUR paints, gives the resultant coatings a permanent hydrophilicity. Furthermore, the polyisocyanates thus modified have an inadequate stability (“pot life”) in aqueous emulsion, while retaining their full NCO activity and reactivity. Accordingly, the time within which the hydrophilic polyisocyanates can be processed is also limited.

There is therefore a desire for hydrophilic polyisocyanates which not only can be incorporated easily into water but also possess a long pot life.

The disadvantage of the above, prior-art, 1K baking systems based on externally blocked polyisocyanates is that the respective blocking agents are eliminated in the course of baking.

It was an object of the present invention, therefore, to provide new, water-dispersible polyisocyanates which can be employed without use of external emulsifiers or employment of high shearing forces and contain no external blocking agents. These new polyisocyanates ought more particularly to produce aqueous emulsions or dispersions having long service lives (“pot life”) and processing lives, while at the same time retaining the full reactivity of the isocyanate groups throughout the service life. The service life of a dispersion or emulsion containing no catalyst ought to amount to at least 8 weeks, preferably at least 12 weeks, at 50° C., during which the latent NCO content must not fall by more than 30%, based on the initial figure.

Surprisingly it has been found that this object can be achieved by means of uretdione-containing modified polyisocyanates with specific internal emulsifiers.

The present invention provides water-dispersible, hydrophilic, uretdione-containing polyisocyanates and polyisocyanate mixtures, a process for preparing them, compositions comprising the hydrophilic polyisocyanates of the invention, and their use as a starting component in the production of polyurethane plastics, more particularly as crosslinkers for water-soluble or water-dispersible film-forming binders or binder components having groups which are reactive towards isocyanate groups.

For the purpose of simplifying the description of the present invention, the term “polyisocyanate” below also stands, synonymously, for mixtures of different polyisocyanates.

By “water-dispersible” in the sense of the invention it is meant that the hydrophilic, water-dispersible polyisocyanates, on contact with water form a fluid within 24 hours that does not show any sign of solid particles to the eye without optical assistants. In order to verify whether a polymer is water-dispersible, 100 mg of the polymer, in the form of a film 100 μm thick, are introduced into 100 ml of water (20° C.) and shaken on a commercially customary shaker bench for 24 hours. If no solid particles can still be discerned after shaking, but the fluid possesses a haze, the polymer is water-dispersible; in the absence of a haze, it is considered “water-soluble”.

The process of the invention for preparing hydrophilic polyisocyanates C comprises the reaction of a prepolymer A, which carries uretdione groups, with at least one emulsifier B, the emulsifier B comprising at least one ionogenic group which in the case of an acidic ionogenic group in water has a pKₐ>8, preferably >10 and very preferably >12 at room temperature or in the case of a basic ionogenic group has a pK_{b} of >8, preferably >10 and very preferably >12 at room temperature.

Polyisocyanates containing uretdione groups are well-known and are described for example in U.S. Pat. No. 4,476,054, U.S. Pat. No. 4,912,210, U.S. Pat. No. 4,929,724 and also EP 417 603. A comprehensive overview of industrially relevant processes for the dimerization of isocyanates to uretdiones is given by J. Prakt. Chem. 336 (1994) 185-200.

The preponderant experience is generally that substances that contain uretdione groups, when introduced in water, suffer a marked degradation in their NCO content after just a few days. For instance, the simplest form of hydrophilization, the attachment of polyether groups, leads to a product which is unstable in water.

Described in DE-A 25 38 484, for example, are one-component dispersions where first of all a prepolymer is prepared from hydroxyl-functional polyesters and polyisocyanates and this prepolymer is reacted off with 30-70 equivalent % of diamines or diols, then hydrophilized and subsequently dispersed. A polyisocyanate employed is the uretdione of isophorone diisocyanate, optionally in mixtures with isophorone diisocyanate and its trimers; in this 1K system, there is a hydroxyl group on each of the two isocyanate groups capped in the uretdione, with the hydroxyl groups being added during dispersing or thereafter. A feature of the hydrophilized polyisocyanate is a very limited stability in water.

DE 10 2005 036654 claims polyurethane dispersions having not only uretdione groups but also groups reactive towards isocyanate groups, in the same molecule. As a result, in addition to the crosslinking between the polyurethane molecules, there is also the possibility of crosslinking within the polymer, referred to as an “intra-penetrating network”. These elimination-free aqueous polyurethane curing agents also have only a very limited storage stability in water.

In accordance with the invention, the conversion of isocyanates to uretdiones tales place preferably in the presence of soluble dimerization catalysts such as, for example, dialkylaminopyridines, trialkylphosphines, phosphoramides or imidazoles. The reaction is carried out optionally in solvents, but preferably in the absence of solvents. When a desired conversion has been reached, the reaction can be stopped by addition of catalyst poisons, though this is not an absolute necessity. Excess monomeric isocyanate is separated off afterwards by short-path evaporation. If the catalyst is sufficiently volatile, the reaction mixture can be freed from the catalyst in the course of monomer removal. In that case there is no need to add catalyst poisons.

For the preparation of polyisocyanates containing uretdione groups, a broad range of isocyanates are suitable in accordance with the invention. Suitable isocyanates are preferably selected from the group consisting of isophorone diisocyanate (IPDI), hexamethylene diisocyanate (HDI), 2,2′-dicyclohexylmethane diisocyanate (2,2′-H₁₂MDI), 2,4′-dicyclohexylmethane diisocyanate 4,4′-dicyclohexylmethane diisocyanate (4,4′-H₁₂MDI), 2-methylpentane diisocyanate (MPDI), 2,2,4-trimethylhexamethylene diisocyanate (2,2,4-TMDI), 2,4,4-trimethylhexamethylene diisocyanate (2,4,4-TMDI), norbornane diisocyanate (NBDI), methylendiphenyl diisocyanate (MDI), toluidine diisocyanate (TDI), tetramethylxylylene diisocyanate (TMXDI) and mixtures thereof. In one preferred embodiment the isocyanate is IPDI and/or 4,4′-H₁₂MDI and/or HDI.

The conversion of these polyisocyanates which carry uretdione groups to prepolymers A containing uretdione groups includes the reaction of the free NCO groups with hydroxyl group-containing monomeric, oligomeric and/or polymeric compounds. The hydroxyl group-containing compound is preferably selected from the group consisting of polyesters, polythioethers, polyethers, polycaprolactams, polyepoxides, polyester amides, polyurethanes, low molecular weight dialcohols, low molecular weight trialcohols, low molecular weight tetraalcohols and monoalcohols. Low molecular weight di-, tri- and/or tetraalcohols are suitable as chain extenders. Chain terminators used can be monoamines and/or monoalcohols, for example as described in EP 669353, EP 669354, DE 3030572, EP 639598 or EP 803524.

Preferred polyesters used are those having an OH number of 30 to 250 mg KOH/g and an average molecular weight of 300 to 6000 g/mol, or monomeric dialcohols, such as, for example, ethylene glycol, propane-(1,2)-diol and propane-(1,3)-diol, 2,2-dimethyl-(1,3)-propanediol, butane-(1,4)-diol, hexane-(1,6)-diol, 2-methylpentane-1,5-diol, 2,2,4-trimethylhexane-(1,6)-diol, 2,4,4-trimethylhexane-(1,6)-diol, heptane-(1,7)-diol, dodecane-(1,12)-diol, octadec-9-ene-(1,12)-diol, thiodiglycol, octadecane-(1,18)-diol, 2,4-dimethyl-2-propylheptane-(1,3)-diol, diethylene glycol, triethylene glycol, tetraethylene glycol, and trans- and cis-1,4-cyclohexanedimethanol.

Preferred prepolymers A containing uretdione groups have a free NCO content of at least 0.2 but not more than 20 wt %, based on the total weight of the prepolymer, and a uretdione groups content of 0.5 to 25 wt %, based on the total weight of the prepolymer, preferably 2 to 20 wt % (calculated as C₂N₂O₂, molecular weight 84). The NCO content is situated preferably in the range from 1.5 to 14 wt %, based on the total weight of the prepolymer.

Besides the uretdione groups, the prepolymer A may also contain isocyanurate, biuret, allophanate, urethane and/or urea structures.

The emulsifier B comprises one or more ionogenic groups, preferably one ionogenic group. Preference is given to an ionogenic group selected from the group consisting of sulphonates and phosphates.

In one preferred embodiment the ionogenic group of the emulsifier B is a radical which gives a predominantly neutral reaction in water. In the case of an acidic ionogenic group it has in water a pKₐ>8 at room temperature or in the case of a basic ionogenic group a pK_{b} of >8 at room temperature.

The emulsifier B may have one or more OH, NH or NH₂ groups or other isocyanate-reactive groups. The number-average molecular weight (Mn) is preferably <1000 and very preferably <500.

The emulsifier B is preferably at least one sulphonic acid selected from the group consisting of hydroxyalkylsulphonic acids, hydroxypolyethersulphonic acids, aminoalkylsulphonic acids and aminopolyethersulphonic acids. Preferred hydroxyalkylsulphonic acids are 2-hydroxyethanesulphonic acids and 3-hydroxypropanesulphonic acids. Preferred aminoalkylsulphonic acids are 2-(cyclohexylamino)ethanesulphonic acid and 3-(cyclohexylamino)propanesulphonic acid. Particularly preferred emulsifiers B are sulphonates containing amino groups.

In a further embodiment the emulsifier B is at least one phosphate.

Suitable neutralizing agents for the acid-group-containing ionogenic groups of the emulsifier B are selected from the group consisting of alkali metal hydroxides and alkaline earth metal hydroxides.

The uretdione-containing prepolymer A and the emulsifier B are mixed with or without solvent and then reacted at suitable temperatures. The solvent is preferably selected from the group consisting of water, acetone, dimethylformamide, N-methylpyrrolidone, ethyl acetate, tetrahydrofuran and dioxane. Preferably the solvent is water.

The process of the invention for preparing hydrophilic polyisocyanates can be carried out optionally in a suitable solvent inert towards isocyanate groups. Suitable solvents are, for example, the customary paint solvents known per se, such as, for example, methoxypropyl acetate, butyl acetate, hydrocarbon-containing aromatics mixtures (Solvesso® from ExxonMobil) and xylene.

While emulsifiers containing amino groups react even at room temperature and give off heat in doing so, in the case of emulsifiers containing hydroxyl groups it is advisable to set a reaction temperature between 40-100 ° C., optionally with use of catalysts known from the literature, such as dibutyltin dilaurate (DBTL), for example.

The ratio of free NCO groups in A to NCO-reactive groups in B prior to their reaction is preferably 2:1 to 1:2, more preferably 1.1:1 to 1:1.1. After the reaction the free NCO content of the hydrophilic polyisocyanate C is preferably <2%, more preferably <1%.

The invention provides reactive compositions which comprise the product C from the reaction of A and B.

The product C is a hydrophilic polyisocyanate, and is also called curing agent C below. With preference the reactive composition of the invention is an aqueous composition.

The hydrophilic polyisocyanate C can be introduced with or without auxiliary solvent, by known methods, into water, to form a dispersion, a suspension, an emulsion or a solution there. The compositions of the invention are stable at 50° C. for at least 8 weeks, preferably at least 12 weeks—in other words, even after 8 or 12 weeks at 50° C., the hydrophilic polyisocyanate C exhibits a reactivity comparable with that of the initial mixture.

Optionally it is possible, prior to the emulsification, to admix the polyisocyanates prepared by the process of the invention with further, non-hydrophilized polyisocyanates, more particularly paint polyisocyanates such as IPDI, HDI, TMDI, H₁₂MDI, and derivatives thereof, e.g. trimers, allophanates, biurets, etc.

The composition of the invention may also preferably comprise the following:

D) a reactant for the hydrophilic polyisocyanate C (curing agent), said reactant containing hydroxyl groups and being dispersible, emulsifiable or soluble in water, and

E) optionally auxiliaries and adjuvants;

F) optional a catalyst F.

Hydroxyl-group-containing components D which are water-soluble, emulsifiable in water or dispersible in water are known in the literature. They may be monomeric, oligomeric and/or polymeric compounds for the component D. Polymeric compounds are preferably selected from the group consisting of polyethers, polyesters, polycarbonates, polyalkyd resins, polyacrylates, polyurethanes and polycaprolactones. The polymeric compounds may be modified using external or internal emulsifiers, so rendering them water-soluble, emulsifiable or dispersible. Monomeric and oligomeric compounds are preferably selected from the group consisting of monomeric diols, triols, tetrols, diamines, triamines and tetramines.

Suitable internal emulsifiers for component D are preferably ionogenic or non-ionogenic building blocks. lonogenic building blocks consist of acids or bases which may additionally contain reactive groups and be neutralized using monomeric bases (e.g. triethylamine) or acids (e.g. acetic acid). Non-ionogenic emulsifiers generally contain relatively long or relatively short polyether units. Such products D are known from the literature and available on the market, and have been described, for example, in DE-A 25 38 484, DE 102005036654 or in Stoye, Freitag, “Lackharze”, Carl Hanser Verlag, Munich Vienna, 1996, or in Meyer-Westhues, “Polyurethane”, Vincentz Network Verlag, Hannover, 2007.

One possibility for preparing resins D with internal emulsifiers is the reaction of suitable alcohol components with a mixture of dimethylolpropionic acid and a di- or polyisocyanate at temperatures of 40-100° C., preferably in an organic solvent, such as acetone, ethyl acetate, dimethylformamide or N-methylpyrrolidone. The organic solvent is optionally removed. Before or after removal of the solvent, the neutralizing agent, triethylamine for example, is added and the reaction product is introduced into water.

As optional component E it is possible to add the customary additives, such as levelling agents, examples being polysilicones or acrylates, light stabilizers, examples being sterically hindered amines, or other auxiliaries, as described in EP 0 669 353, for example, examples being stabilizers, degassing agents, emulsifying assistants and dispersing additives, in a total amount of 0.05 to 5 wt %, based on the total weight of the composition. Fillers and pigments such as titanium dioxide, for example, can be added in an amount of up to 50 wt % of the total composition. Preference is given to auxiliaries and additives that are customary in the paints sector, such as, for example, levelling assistants, colour pigments, fillers, matting agents and external emulsifiers.

Organic solvents are suitable as auxiliary E. Liquid substances which do not react with other ingredients may be added to the aqueous composition of the invention, examples being ethyl acetate, butyl acetate, ethylene glycol monomethyl ether acetate, ethylene glycol monoethyl ether acetate, 1-methoxyprop-2-yl-acetate, 3-methoxy-n-butyl acetate, acetone, 2-butanone, 4-methyl-2-pentanone, cyclohexanone, toluene, xylene, chlorobenzene, white spirit, aromatics with relatively high degrees of substitution, of the kind available commercially, for example, under the names Solvent naphtha, Solvesso®, Isopar®, Nappar® (Deutsche EXXON CHEMICAL GmbH) and Shellsol® (Deutsche Shell Chemie GmbH), carbonic esters, such as dimethyl carbonate, diethyl carbonate, 1,2-ethylene carbonate and 1,2-propylene carbonate, lactones, such as propiolactone, butyrolactone, caprolactone and methylcaprolactone, and also solvents such as propylene glycol diacetate, diethylene glycol dimethyl ether, dipropylene glycol dimethyl ether, diethylene glycol ethyl ether acetate, diethylene glycol butyl ether acetate, N-methylpyrrolidone and N-methylcaprolactam or any desired mixtures of such solvents.

These solvents are preferably used merely as auxiliary solvents and account for only relatively small fractions as compared with the principal solvent, water. The water:auxiliary solvent volume ratio is preferably greater than 2:1 and is preferably situated in the range from 100:1 to 10:1.

In one preferred embodiment, for the preparation of the composition of the invention, the aqueous, uretdione-containing curing agent C is mixed with the aqueous resin D and admixed optionally with further auxiliary and adjuvant components E. In another embodiment, for preparing the composition of the invention, one or more of the non-aqueous components C and/or D and optionally E is or are introduced into one of the aqueous components C and/or D and optionally E. In a further embodiment, for preparing the composition of the invention, all the non-aqueous components C, D and optionally E are introduced successively or simultaneously into water.

The composition of the invention is stable at 50° C. for at least 8 weeks, preferably after 12 Weeks—that is, even after 8 or 12 weeks at 50° C., it exhibits a reactivity substantially the same as that of the initial mixture. The latent NCO loss of the aqueous composition of the invention after storage at 50° C. for 8 weeks, preferably after storage at 50° C. for 12 weeks, is not more than 30%, based on the initial figure. These figures are valid for compositions which have no catalyst F for increasing the reactivity.

Besides, C, D and optionally E, the composition of the invention may also comprise catalysts or catalyst mixtures F (hereinafter catalysts F).

Catalysts F are added to the composition to raise the reactivity of uretdione groups and have become known in particular for powder coatings.

Suitable catalysts F are preferably F1 and/or F2,

F1 being selected from the group consisting of metal acetylacetonates, preferably zinc acetylacetonates, optionally in combination with tetraalkylammonium halides or tetraalkylphosphonium halides and acid-scavenging components, examples being epoxides, carbodiimides, aziridines, oxazolines or hydroxyalkylamides, and

F2 being selected from the group consisting of tetraalkylammonium hydroxides, tetraalkylphosphonium hydroxides and carboxylates, optionally in combination with epoxides. F2 is preferably a tetraalkylammonium carboxylate, more preferably a tetraalkylammonium benzoate.

For reactivity adjustment of F it is possible to add mono-, di- or poly acids, preferably carboxylic acids, an example being oxalic acid, to the reaction mixture.

Customary catalysts known from polyurethane chemistry can also be used, examples being tertiary amines such as triethylamine, pyridine, methylpyridine, benzyldimethylamine, N,N-endoethylenpiperazine, N-methylpiperidine, pentamethyldiethylenetriamine, N,N-dimethylaminocyclohexane, N,N′-dimethylpiperazine or metal salts such as iron(II) chloride, aluminium tri(ethylacetoacetate), zinc chloride, zinc(II) n-octanoate, zinc(II) 2-ethyl-1-hexanoate, zinc(II) 2-ethylcaproate, zinc(II) stearate, zinc(II) naphthenate, zinc(II) acetylacetonate, tin(II) n-octanoate, tin(II) 2-ethyl-1-hexanoate, tin(II) ethylcaproate, tin(Il) laurate, tin(II) palmitate, dibutyltin(IV) oxide, dibutyltin(IV) dichloride, dibutyltin(IV) diacetate, dibutyltin(IV) dimaleate, dibutyltin(IV) dilaurate, dioctyltin(IV) diacetate, molybdenum glycolate or any desired mixtures of such catalysts.

In the process of the invention the catalysts F can be used with a concentration in the range from 0.001 to 2 wt %, preferably in the range from 0.005 to 0.5 wt %, based on the total weight of the reactants.

The combination of C, D and F and also optionally E exhibits an increased reactivity in comparison with the variant without F. The reactivity and also the storage stability are dependent, however, on the composition and the concentration of component F. The storage stability, however, is at least 8 weeks, preferably at least 12 weeks, at room temperature (20° C.). The reason for the lower storage stability in comparison with catalyst-free compositions is that the catalyst raises the reactivity and hence reduces the storage stability. Nevertheless, the storage stability of the composition of the invention with catalyst is still higher than that of prior-art comparative products which likewise include a catalyst.

Additionally provided by the invention is the use of the polyisocyanates of the invention as starting components in the production of polyurethane plastics, more particularly as crosslinkers for water-soluble or dispersible film-forming binders or film-forming binder components in the production of coatings using aqueous coating materials based on such binders or binder components.

Also provided by the invention, finally, is the use of these polyisocyanates as starting components in the production of blocked polyisocyanates which are water-dispersible or are present in dispersion in water.

The polyisocyanates of the invention represent valuable starting materials for the production of polyurethane plastics by the isocyanate polyaddition method.

The invention accordingly also provides coatings, adhesive bonds and sealants produced using a composition comprising at least one polyisocyanate of the invention, and also a method for producing them. The at least one polyisocyanate is preferably prepared by means of the process of the invention. With particular preference the coatings, adhesive bonds and sealants of the invention are produced using a composition of the invention.

The method for producing a coating, adhesive bond or a sealant comprises the application to a substrate of a composition comprising at least one polyisocyanate of the invention and/or at least one polyisocyanate prepared in accordance with the invention. The applied composition may comprise further components such as those specified above.

The curing and drying of the composition is carried out preferably at temperatures between 80° C. and 240° C. in a time between 30 s and one week. With particular preference the curing and drying is carried out at 120-200° C. in a time of 5 minutes to one hour.

In one embodiment the coatings, adhesive bonds and sealants of the invention, after appropriate drying at 100-240° C., preferably at 120-200° C., more preferably at 140-180° C., have good technical paint properties.

Substrates contemplated for application of the composition of the invention thereto include any desired substrates, such as, for example, metal, wood, glass, stone, ceramic materials, concrete, rigid and flexible plastics, textiles, leather and paper, which prior to said application may optionally also be provided with customary primers.

Besides the preferred use as crosslinker components for aqueous 1K PUR paints, the compositions of the invention are outstandingly suitable as crosslinkers for aqueous dispersion-based adhesives, leather coatings and textile coatings or textile printing pastes, as papermaking assistants free from organically bonded halogens (AOX-free), or else as additives for mineral building materials, examples being concrete or mortar compositions.

The reactive curing agents C and compositions of the invention, processes for preparing them and their use are described by way of example below, without any intention that the invention should be confined to these exemplary embodiments.

Where reference is made below to ranges, general formulae or classes of compound, the intention is that these should encompass not only the corresponding ranges or groups of compounds which are explicitly mentioned, but also all sub-ranges and sub-groups of compounds that can be obtained by extraction of individual values (ranges) or compounds. Where documents are cited in the context of the present description, the intention is that their content should belong in full to the disclosure content of the present invention.

The examples which follow are intended to elucidate the invention in more detail. The present invention is described by way of example, without any intention that the invention—the scope of application of which is evident from the whole of the description and the claims—should be confined to the embodiments specified in the examples.

### EXAMPLES

### Example a

### Preparation of a Neutral, Non-Ionogenically Stabilized Uretdione (Not Inventive)

223.9 g of IPDI uretdione (see example c1) were dissolved with 300.9 g of Polyglycol M350 (polyglycol monomethyl ether, Clariant) and 0.3 g of dibutyltin dilaurate (Aldrich) in 250 ml of acetone. After 15 hours under reflux, the solution was cooled. 250 g of this product were admixed with 262.5 g of fully demineralized water, with vigorous stirring in a dispermat (3000 rpm). The acetone was removed on a rotary evaporator at 40 mbar and 60° C., after which the product was filtered using a 50 μm filter. The latent NCO content was 4.59%, the solids about 46% and the viscosity 200 mPas.

After 8 weeks at 20° C. this aqueous dispersion had a latent NCO content of 3.0% and after 8 weeks at 50° C. a latent NCO content of 1.26%. The hydrophilization of uretdione-containing curing agents with polyethers therefore does not produce a storage-stable aqueous dispersion.

### Example b

### Preparation of a Non-Neutral, Ionogenically Stabilized Uretdione (Not Inventive)

307.6 g of IPDI uretdione (see example c1) were dissolved in 180 ml of acetone and 0.3 g of dibutyltin dilaurate (Aldrich) was added. 112.4 g of dimethylaminopropylamine (DMAPA, Aldrich) were carefully added dropwise and the temperature was held at below 30° C. by cooling. After a subsequent reaction time of 1 hour, 200 g of this product were first neutralized with 23.8 g of acetic acid and then admixed with 163.8 g of fully demineralized water with vigorous stirring in a dispermat (3000 rpm). The acetone was removed on a rotary evaporator at 40 mbar and 60° C. after which the product was filtered using a 50 pm filter. The latent NCO content was 0.55% (theoretical 4.17%), the solids was about 46% and the viscosity was 200 mPas. Right at the start, therefore, the product does not have the expected NCO content. The hydrophilization of uretdione-containing curing agents with tertiary amino alkyl amines does not produce a storage-stable aqueous dispersion.

### Example c

### Preparation of the Uretdione-Containing Curing Agent C

c1) Preparation of a Uretdione Prepolymer from IPDI (Not Inventive)

Uretdione based on IPD1 was prepared in accordance with the instructions from DE 10 2005 036654. 10 000 g (45.0 mol) of isophorone diisocyanate (Vestanat IPDI, Evonik) were admixed with 200 g (2%) of 4-dimethylaminopyridine (DMAP) as catalyst at room temperature under dry nitrogen and with stirring. After 24 hours the reaction mixture, which had an NCO content of 27.2%, corresponding to a degree of oligomerization of 26.5%, was free from volatile constituents, without prior addition of the catalyst poison using a thin-film evaporator at a temperature of 160° C. and a pressure of 0.3 mbar. This gave a highly viscous, pale yellow-coloured uretdione polyisocyanate having a free NCO group content of 16.8% and a monomeric IPDI content of 0.3%. No isocyanurate structures were found in the ¹³C NMR spectrum.

c2) Preparation of the Curing Agent C (Inventive)

1050.6 g of IPDI uretdione (from preparation procedure c1) were dissolved with 95.5 g of trimethylolpropane (Aldrich) and 0.23 g of DBTL (dibutyltin dilaurate, Aldrich) in 1.4 l of acetone. After an hour of stirring under reflux (free NCO content: 3.64%), 103 g of butanol were added and heating under reflux was continued for 1.5 hours more, after which the free NCO content was 1.49%. After cooling, 151 g of Vestamin A95 (sodium hydroxide-neutralized, amine-containing alkylsulphonate, 50% in water, Evonik) were added dropwise as internal emulsifier. After the end of addition, heating at reflux was repeated for 1.5 hours and the product was then cooled. The free NCO content was 0.1%, the free amine number 0.2%.

650 g of this product were admixed with 604 g of demineralized water with vigorous stirring in a dispermat (3000 rpm). The acetone was removed on a rotary evaporator at 40 mbar and 60° C. and the product was subsequently filtered using a 50 μm filter. The latent NCO content was 4.5%, the solids about 27% and the viscosity 163 mPas.

### Example d

### Preparation of the Water-Based Resin D (Not Inventive)

829.5 g of Vestanat H₁₂MDI (dicyclohexylmethylene diisocyanate, Evonik) and 169.4 g of dimethylolpropionic acid (Aldrich) were dissolved with 0.5 g of DBTL (dibutyltin dilaurate, Aldrich) in 1.1 l of acetone. After 12 hours under reflux, the batch was cooled and 57 g of 1,4-butanediol (Aldrich), 1363 g of Oxyester T1136 (liquid hydroxyl polyester with OHN:

112, Evonik), 169 g of trimethyloipropane (Aldrich) and 0.8 g of DBTL (dibutyltin dilaurate, Aldrich) were added. After a further 18 hours of stirring under reflux, the NCO content had dropped to 0.1%. The acid number was 27 and the OH number was 82, based in each case on the solids.

250 g of this product were neutralized with 7.6 g of dimethylaminoethanol and admixed with 8.2 g of a 10% strength solution of adipic hydrazide (Aldrich) in water. Then 345 g of fully demineralized water were added in a dispermat with vigorous stirring (3000 rpm). At the end a drop of triethylamine was added as well. The acetone was thereupon removed on a rotary evaporator at 40 mbar and 60° C., after which the product was filtered using a 50 μm filter. The solids was about 35%, the viscosity 190 mPas.

### Example e

### Storage Stability and Reactivity

e1) Storage of the Curing Agent

The curing agent C (latent NCO content 4.9%) was stored at 50° C. for 8 weeks. The stored variant is hereinafter abbreviated to C(g). The curing agent C(g) showed a latent NCO content of 4.6%.

e2) Storage of the Mixtures: 70.4 g of C +29.6 g of D

| | Initial value | 8 w @ 50° C. |
|---|---|---|
| NCO content | 1.2% | 0.9% |
| Curing 30 min 180° C. | | |
| Erichsen cupping [mm] | >10 | >10 |
| Cross-cut [0-5] | 0 | 0 |
| MEK resistance [DR] | >100 | >100 |

e3) Mixture with Stored Curing Agent 70.4 g of C(g)+29.6 g of D

After the mixing of the stored curing agent C with D, the mixture is not stored any more. Here, therefore, only the initial figure was measured.

| | Value with stored curing agent C |
|---|---|
| NCO content | 1.1% |
| Curing 30 min 180° C. | |
| Erichsen cupping [mm] | >10 |
| Cross-cut [0-5] | 0 |
| MEK resistance [DR] | >100 |

Even after 8 weeks at 50° C. the reactivity is sufficient, irrespective of whether only uretdione-containing curing agent (e3) or the mixture as a whole (e2) was stored.

### Example f

### Catalyzed Systems

Owing to the catalyst, catalyzed systems possess a higher reactivity than the uncatalyzed systems tested in the examples indicated above. As a consequence of this they are also not so storage-stable, but cure even at much lower temperatures, which is more favourable for certain applications. The storage stability was therefore measured after curing at 130° C. for 30 minutes.

### f1) Comparative Example

163.2 g of the curing agent C from example c2) was mixed with stirring with 15 g of Voranol CP 450 (hydroxyl-containing polyether, Dow), 3.75 g of Poly G 55-37 (hydroxyl-containing polyether, Arch Chemicals), 5 g of fully demineralized water, 2 g of Byk 011 (additive, Byk-Chemie). This was followed by additions of 0.4 g of Byk 348 and 0.1 g of Byk 333 (additives, Byk-Chemie).

### f2) Inventive Example

The mixture f1) was admixed with 3.62 g of 50% strength tetraethylammonium benzoate solution (catalyst, Sachem) and with 4.8 g of Polypox 1E700 8W (activator, hydrophilized bisphenol A diepoxide, UPPC, Dow).

Curing (30 min 130° C.)

| | f1* | f2 |
|---|---|---|
| Pendulum hardness [s] | 53 | 193 |
| MEK resistance [DR] | 1-2 | >100 |

* f1 is comparative example

Storage of f2

| | Initial value | 8 w at RT |
|---|---|---|
| Pendulum hardness [s] | 193 | 204 |
| MEK resistance [DR] | >100 | >100 |

Even after 8 weeks at room temperature the reactivity is unchanged in the case of f2.

The measurement values were determined as follows:

Erichsen cupping to DIN 53 156

Ball impact to ASTM D 2794-93

Pendulum hardness to DIN 53 157

Cross-cut to DIN 53 151

MEK resistance:

The methyl ethyl ketone resistance test is carried out by rubbing with an absorbent cotton ball soaked in methyl ethyl ketone under a weight of 1 kg until the coat dissolves. In this rubbing procedure, double rubs (DR) are counted. A figure of >100 therefore means that over more than 100 double rubs there was still no dissolution of the coat observable.

1-15. (canceled)

16. A process for preparing hydrophilic polyisocyanates, comprising:
reacting a prepolymer A which carries uretdione groups, with at least one emulsifier B,
the emulsifier B comprising at least one ionogenic group which in the case of an acidic ionogenic group in water has a pKₐ>8 at room temperature or in the case of a basic ionogenic group has a pK_{b}>8 at room temperature;
wherein the emulsifier B is at least one sulphonate selected from the group consisting of hydroxyalkylsulphonates, hydroxypolyethersulphonates, aminoalkylsulphonates and aminopolyethersulphonates.

17. The process according to claim 16, further comprising reacting a polyisocyanate which carries uretdione groups with monomeric, oligomeric and/or polymeric compounds which contain hydroxyl groups, the prepolymer A being obtained and the compound which contains hydroxyl groups being selected from the group consisting of polyesters, polythioethers, polyethers, polycaprolactams, polyepoxides, polyesteramides, polyurethanes, low molecular weight dialcohols, low molecular weight trialcohols, low molecular weight tetraalcohols and monoalcohols.

18. The process according to claim 16, the prepolymer A having a free NCO content in the range from 0.2 wt % to 20 wt %, based on the total weight of the prepolymer A, and having a uretdione group content of 0.5 to 25 wt %, based on the total weight of the prepolymer A.

19. A hydrophilic polyisocyanate having at least one uretdione group, the polyisocyanate comprising at least one internal emulsifier preparable by a process according to claim 16.

20. A composition, comprising:
C at least one hydrophilic polyisocyanate according to claim 19, and optionally water.

21. The composition according to claim 20, further comprising
D a reactant for the hydrophilic polyisocyanate C, said reactant containing hydroxyl groups and being dispersible or emulsifiable or soluble in water, and optionally
E an auxiliary and/or adjuvant, and optionally
F a catalyst.

22. The composition according to claim 21, the reactant D being selected from the group consisting of polyethers, polyesters, polycarbonates, polyalkyd resins, polyacrylates, polyurethanes and polycaprolactones.

23. A polyurethane plastic, comprising: the hydrophilic polyisocyanate according to claim 19.

24. A crosslinker for water-soluble or water-dispersible film-forming binders, adhesives binders or sealant binders or binder components having groups which are reactive towards isocyanate groups, comprising: the hydrophilic polyisocyanate according to claim 19.

25. A polyurethane plastic, comprising: the composition according to claim 20.

26. Water-soluble or water-dispersible film-forming binders, adhesives binders or sealant binders or binder components having groups which are reactive towards isocyanate groups comprising: the composition according to claim 20.

27. A coating, adhesive bond or sealant produced using a composition comprising a polyisocyanate according to claim 19.

28. A method for producing a coating, adhesive bond or sealant, comprising applying a composition according to claim 20 to a substrate.
